(19) United States
(12) Patent Application Publication
(10) Pub. No.: US 2015/0051295 A1
(43) Pub. Date: Feb. 19, 2015

(54) USE OF PROPANOL AND PROPENOL DERIVATIVES AS ANTIOXIDANTS

(71) Applicant: MERCK PATENT GmbH, Darmstadt (DE)

(72) Inventors: Thomas Rudolph, Darmstadt (DE); Rene Peter Scheurich, Gross-Zimmern (DE)

(73) Assignee: MERCK PATENT GMBH, Darmstadt (DE)

(21) Appl. No.: 14/348,961

(22) PCT Filed: Sep. 27, 2012
(86) PCT No.: PCT/EP2012/004061

§ 371 Date: Apr. 1, 2014

(30) Foreign Application Priority Data
Oct. 1, 2011 (DE) 10 2011 114 780.6

(51) Int. Cl. A61K 8/34; A61Q 19/00; A61Q 5/12; A61Q 5/00; A61Q 1/10

(52) U.S. Cl. CPC A61K 8/34; A61Q 5/00; A61Q 1/10; A61Q 5/12; A61Q 19/00; A61K 2800/5922
USPC 514/720; 568/646; 568/644; 514/730; 514/721

(57) ABSTRACT

The invention relates to the use of propanol and propenol derivatives as antioxidants, to compositions comprising propanol and propenol derivatives and the preparation thereof, and to certain propanol and propenol derivatives and the preparation thereof.

The invention relates to the use of propanol and propenol derivatives as antioxidants, to compositions comprising propanol and propenol derivatives and to preparation thereof, and to certain propanol and propenol derivatives and the preparation thereof.

The object of care cosmetics is wherever possible to maintain the impression of youthful skin. In principle, there are various ways of achieving this object. Thus, existing skin damage, such as irregular pigmentation or wrinkling, can be compensated for by hiding powders or creams. Another approach is to protect the skin against environmental influences which lead to permanent damage and thus ageing of the skin.

Thus, for example, with increasing age the skin loses the ability to absorb free radicals generated by external or internal stress factors. This weakening defence function of the skin against harmful influences can be compensated for, for example, by the external supply of substances which are capable of replacing the diminishing defence or repair functions. Such substances are, for example, substances which have an antioxidative action and thus reduce oxidative stress of the skin, or are able to preclude this in a preventative manner.

The object of the present invention was therefore the provision of substances which can be used as antioxidants, thus have a protective action against oxidative stress and are able to improve the general condition of the skin or hair.

Surprisingly, it has now been found that certain propanol and propenol derivatives have excellent properties as antioxidants.

Selected propanol and propenol derivatives are known in the literature. Thus, for example, the preparation of differently substituted 1,3-diphenyl-1-propanol derivatives is disclosed in Kose et al, 2010, Org. Biomol. Chem. 8, 896-900.

The synthesis of differently substituted 1,3-diphenyl-1-propenol derivatives is described by way of example in Sakurai et al, 2009, Chem. Pharm. Bull. 57(5), 511-512.

However, the prior art does not describe the antioxidative action of these compounds.

The present invention therefore relates firstly to the use of one or more compounds of the formula (1) and/or (2)

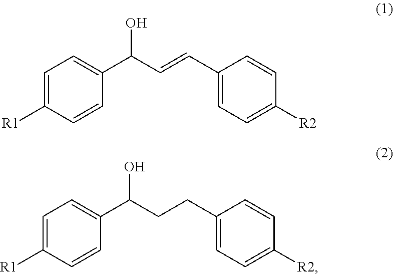

where R1 and R2 stand, independently of one another, for hydrogen, C1- to C12-alkoxy or C1- to C12-alkyl, as antioxidant.

The structural formula (1) depicted encompasses in accordance with the invention in each case both the trans and the corresponding cis isomers, which can be formed, for example, photochemically from the trans isomers.

Thus, the compounds of the formula (1) and/or (2) can be used for prophylaxis against or for combating of oxidative stress.

The reduction of oxidative stress results in a number of positive effects: thus, compounds of the formula (1) and/or (2) can also be used for the care, preservation or improvement of the general condition or appearance of the skin or hair, or for prophylaxis against or for combating of time- and/or light-induced ageing processes of the skin or hair.

In particular, this can be, for example, prophylaxis against or the reduction of dry skin, wrinkling and/or pigment defects, and/or damaging effects of UV rays on the skin, and prophylaxis against or the reduction of skin unevenness, such as wrinkles, fine lines, rough skin or large-pored skin.

The antioxidative action of the compounds also facilitates prophylaxis against or combating of sebum oxidation and/or acne. Compounds of the formula (1) and/or (2) thus also have an anticomedogenic or anti-acne action.

The compounds can also be used for the stabilisation of other formulation constituents, such as UV filters, dyes and vitamins.

In particular, the use of the compounds of the formulae (1) and/or (2) gives, for example, the following advantages:
antioxidant action against free radicals, which are induced, for example, by UV light or by thermolytic processes, such as smoking, such as, for example, against the superoxide free-radical anion or the NO free radical, or against reactive oxygen species, such as, for example, against singlet oxygen and peroxides;
strong antioxidative activity in combination with high molecular stability against environmental influences, such as heat or light, in particular UV light, but also infrared and visible radiation;
product-stabilising action on cosmetic, pharmaceutical, in particular dermatological products, in particular those which comprise dyes, consistency factors, fragrances or vitamins;
compounds of the formulae (1) and (2) are suitable for DNA protection. In addition, they can promote cell proliferation and cell differentiation in the skin and thus contribute, for example, to the anti-ageing, antiwrinkle and wound-healing action;
compounds of the formulae (1) and (2) are suitable for the generation or increase (“boost” effect) of light protection factors, such as LSF, SPF, PPD or IPD, or free-radical protection factors;
stabilising action on auto-oxidisable polyethylene glycol (PEG) or polyglycerol (PG) derivatives, such as, in particular, PEG- or PG-containing emulsifiers, as mentioned below in this application, or a reduction in the damaging effect of degradation products of auto-oxidisable polyethylene glycol (PEG) or polyglycerol (PG) derivatives;
stabilising action on dyes, consistency factors or fragrances, or on antioxidants or vitamins, and UV filters and titanium dioxide-containing pigments, in particular in cosmetic, pharmaceutical, in particular dermatological products;

Further advantages of the compounds of the formulae (1) and (2) are their inexpensive accessibility and their advantageous pharmaceutical formulation properties: the compounds do not crystallise out if they have been incorporated into preparations, have no inherent coloration and are odour-neutral.

The radical R1 in the compounds of the formula (1) and (2) preferably stands for hydrogen, C1- to C6-alkyl or C1- to C6-alkoxy; particularly preferably for hydrogen, C3- to C5-alkyl or C1- to C2-alkoxy; very particularly preferably for hydrogen, methoxy, isopropyl or t-butyl.

The radical R2 in the compounds of the formula (1) and (2) preferably stands for hydrogen, C1- to C6-alkyl or C1- to C6-alkoxy; particularly preferably for hydrogen, C3- to C5-alkyl or C1- to C2-alkoxy; very particularly preferably for hydrogen, methoxy, isopropyl or t-butyl.

Compounds of the formula (1) which are particularly preferred for the use according to the invention are selected from compounds of the formula (1a) to (1p), very particular preference is given to the compounds (1a), (1b) and (1l), in particular (1a).

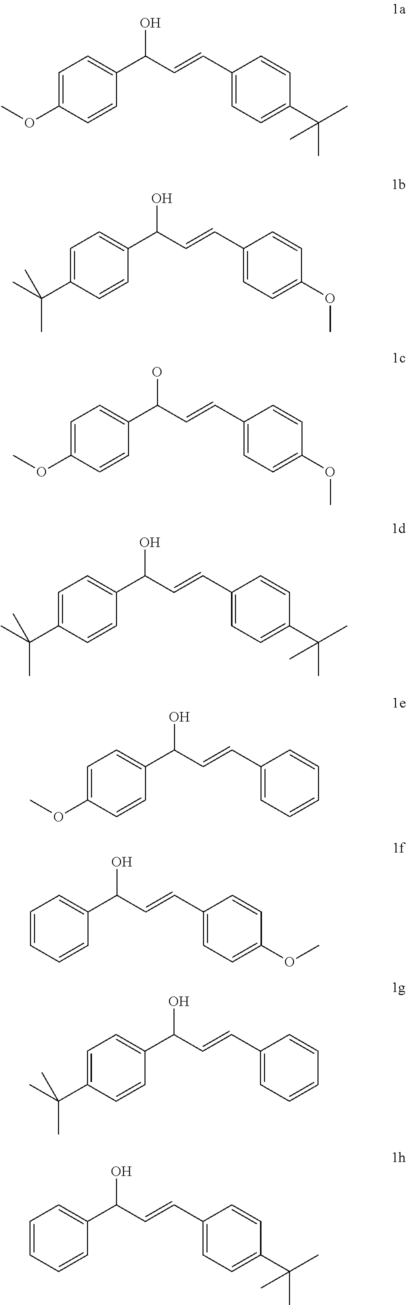

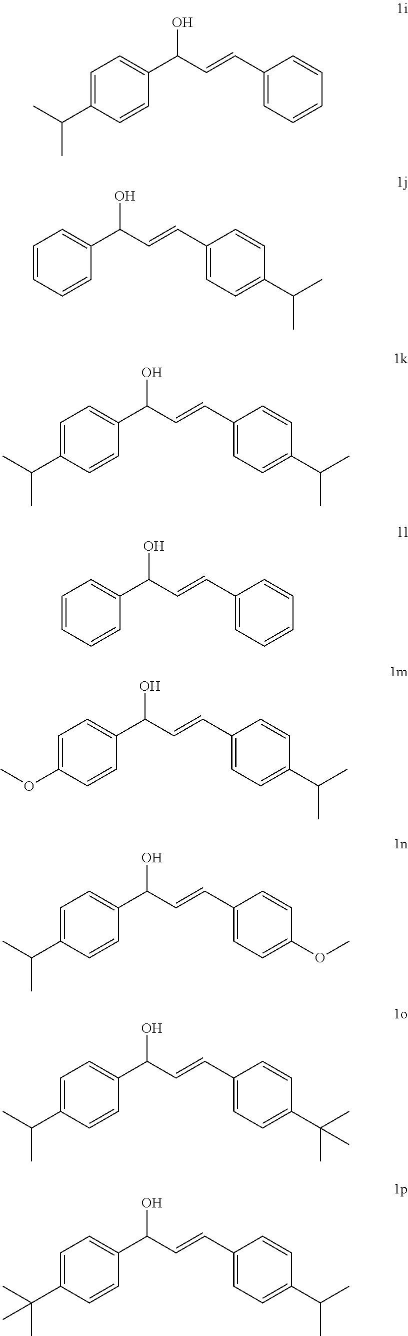

Compounds of the formula (2) which are particularly preferred for the use according to the invention are selected from compounds of the formula (2a) to (2p), very particular preference is given to the compounds (2a), (2b) and (2l), in particular (2a).

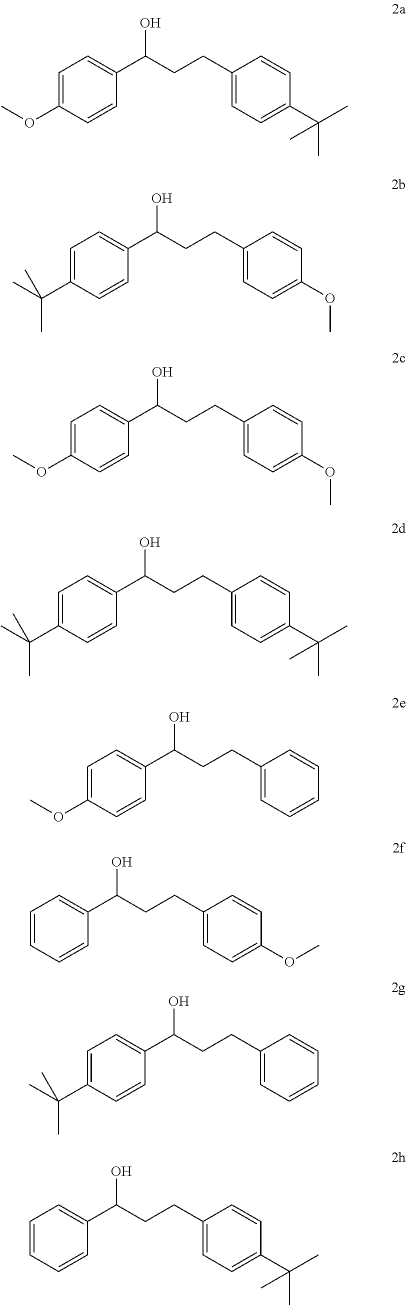

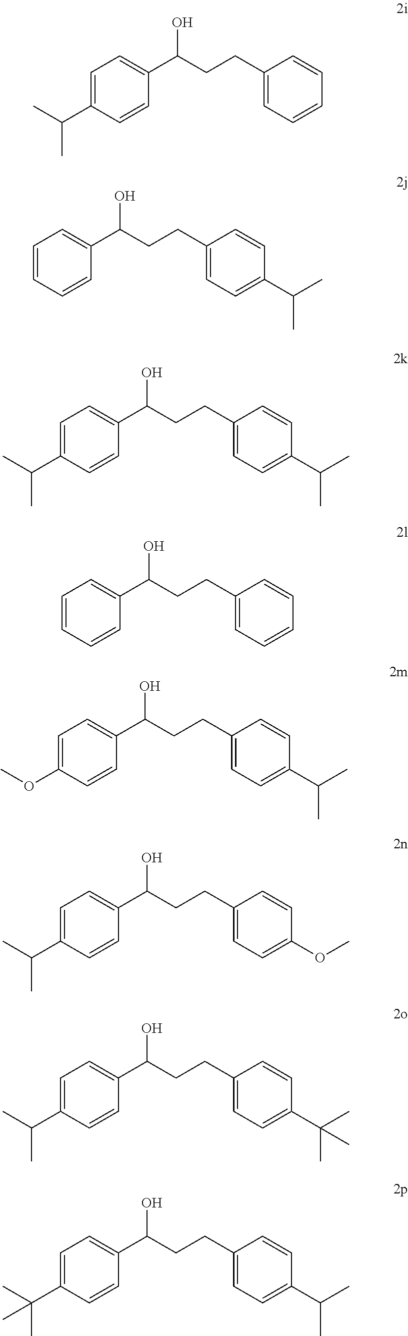

The compounds of the formula (1) and (2) can be prepared by catalytic hydrogenation from the respective corresponding chalcone (1-A) or dihydrochalcone (2-A) of the formula

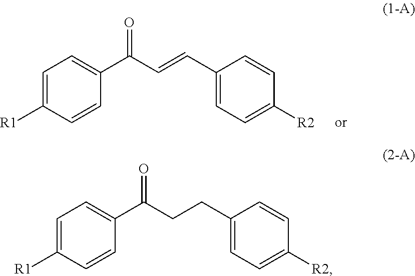

where the radicals R1 and R2 are as defined above for formula (1) or formula (2).

Furthermore, compounds of the formula (2) can also be prepared by catalytic hydrogenation from the chalcone (1-A).

The possible preparation routes are illustrated in the two schemes below, where the synthesis of the compounds of the formula (1l) (Scheme 1, first route) or (2l) (Scheme 1, first route; Scheme 2) is depicted by way of example. The reaction conditions can be obtained from the schemes.

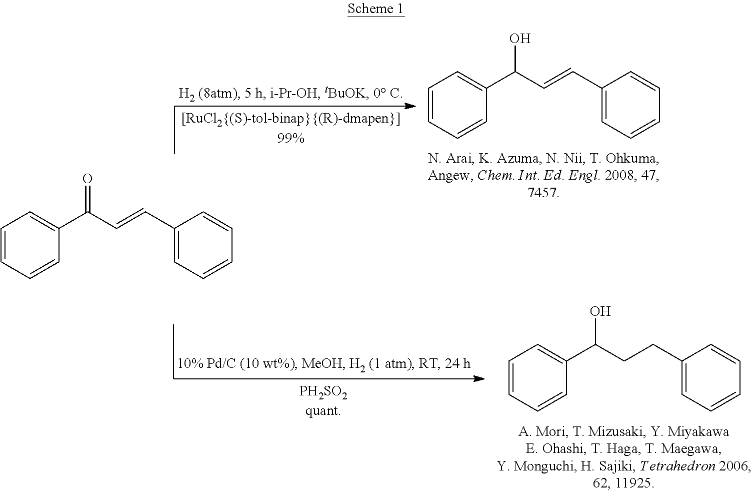 [SMILES: OC(/C=C/c1ccccc1)c1ccccc1 ; O=C(/C=C/c1ccccc1)c1ccccc1 ; OC(CCc1ccccc1)c1ccccc1]

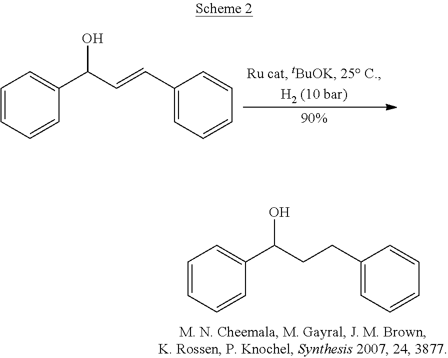 [SMILES: OC(/C=C/c1ccccc1)c1ccccc1 ; OC(CCc1ccccc1)c1ccccc1]

The starting compounds of the formula (1-A) and (2-A) can be obtained, for example, by the preparation route illustrated in Scheme 3:

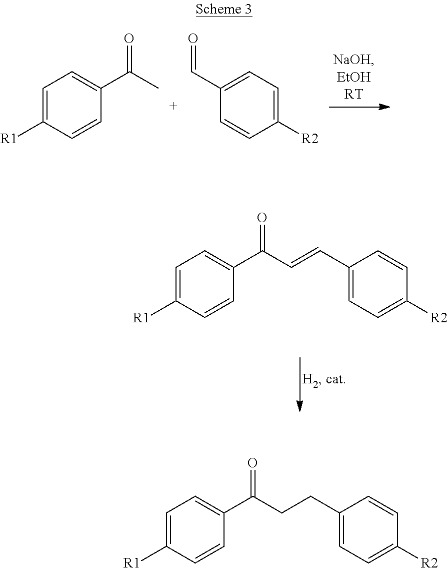

All other starting materials are commercially available or can be prepared in a simple manner which is known to the person skilled in the art.

In general, the person skilled in the art is presented with absolutely no difficulties in selecting the suitable reaction conditions, such as temperature, solvents or reaction time.

In accordance with the present invention, alkyl stands as abbreviation for alkyl group. The alkyl group may be straight-chain or branched.

In accordance with the invention, a C1- to C12-alkyl is taken to mean, for example, methyl, ethyl, isopropyl, propyl, butyl, sec-butyl or tert-butyl, pentyl, 1-, 2- or 3-methylbutyl, 1,1-, 1,2- or 2,2-dimethylpropyl, 1-ethylpropyl, hexyl, heptyl, 1-ethylpentyl, octyl, 1-ethylhexyl, nonyl, decyl, undecyl or dodecyl.

A C1- to C6-alkyl is taken to mean, for example, methyl, ethyl, isopropyl, propyl, butyl, sec-butyl or tert-butyl, pentyl, 1-, 2- or 3-methylbutyl, 1,1-, 1,2- or 2,2-dimethylpropyl, 1-ethylpropyl or hexyl.

A C3- to C5-alkyl is, for example, isopropyl, propyl, butyl, sec-butyl or tertbutyl, pentyl, 1-, 2- or 3-methylbutyl, 1,1-, 1,2- or 2,2-dimethylpropyl or 1-ethylpropyl.

Alkoxy stands as abbreviation for alkoxy group. The alkoxy radical may be linear or branched.

Examples of C1- to C12-alkoxy groups are furthermore methoxy, ethoxy, propoxy, isopropoxy, butoxy, isobutoxy or tert-butoxy, 1,1-, 1,2- or 2,2-dimethylpropoxy, 1-ethylpropoxy, hexoxy, heptoxy, 1-ethylpentoxy, octoxy, nonoxy, decoxy, undecoxy, dodecoxy, tridecoxy, tetradecoxy, pentadecoxy, hexadecoxy, heptadecoxy, octadecoxy or nonadecoxy.

C1- to C6-alkoxy stands, for example, for methoxy, ethoxy, propoxy, isopropoxy, butoxy, isobutoxy or tert-butoxy, 1,1-, 1,2- or 2,2-dimethylpropoxy, 1-ethylpropoxy or hexoxy.

In accordance with the invention, C1- to C2-alkoxy is taken to mean methoxy or ethoxy.

The present invention also relates to a preparation comprising at least one compound of the formula (1) and/or (2)

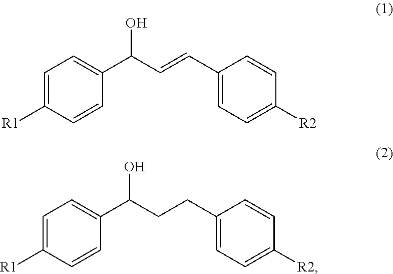

where R1 and R2 stand, independently of one another, for hydrogen, C1- to C12-alkoxy or C1- to C12-alkyl,
and at least one vehicle which is suitable for topical applications.

R1 and R2 preferably stand for radicals as defined above. The compounds of the formulae (1) and (2) are particularly preferably selected here from the compounds of the formula (1a) to (1p) and (2a) to (2p), as defined above, very particularly preferably from the compounds of the formula (1a), (1b), (1l), (2a), (2b) and (2l), in particular from the compounds of the formula (1a) and (2a).

The substances of the present invention can also be combined with one another in the formulations.

The preparations here are usually preparations which can be applied topically, for example cosmetic or dermatological formulations or medical products. In this case, the preparations comprise a cosmetically or dermatologically suitable vehicle and, depending on the desired property profile, optionally further suitable ingredients. In the case of pharmaceutical preparations, the preparations in this case comprise a pharmaceutically tolerated vehicle and optionally further pharmaceutical active compounds.

In the sense of the present invention, the term composition or formulation is also used synonymously alongside the term preparation.

The preparations may include or comprise, essentially consist of or consist of the said requisite or optional constituents. All compounds or components which can be used in the preparations are either known and commercially available or can be synthesised by known processes.

The preparation is preferably a cosmetic or pharmaceutical preparation; the preparation is particularly preferably a cosmetic preparation.

The present invention also relates to a process for the preparation of a preparation, as described above, characterised in that the at least one compound of the formula (1) and/or (2) is mixed with the vehicle which is suitable for topical applications and optionally with further active substances or assistants. Suitable vehicles and active substances or assistants are described in detail in the following part.

In preferred embodiments, the at least one compound of the formula (1) and/or (2) is typically employed in the preparations according to the invention in amounts of 0.01 to 20% by weight, preferably in amounts of 0.05 to 10% by weight, particularly preferably in amounts of 0.1 to 5% by weight and very particularly preferably in amounts of 0.5 to 2% by weight. The person skilled in the art is presented with absolutely no difficulties here in selecting the amounts correspondingly depending on the intended action of the preparation.

The addition of compounds, as disclosed in WO 2007/121818 A1, to the formulations according to the invention is particularly advantageous since this facilitates mutual action enhancement. A possible embodiment of the present invention is therefore also preparations which additionally comprise one or more compounds of the formula (3)

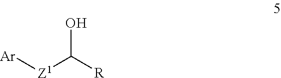

where R stands for

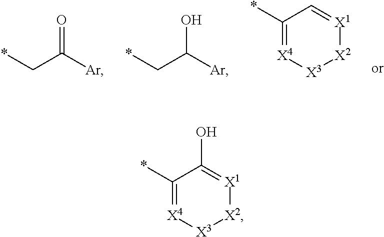

where
Ar in each case, independently of one another, stand for an unsubstituted or mono- or polysubstituted aromatic ring or condensed ring systems having 6 to 18 C atoms, of which at least one ring has aromatic character, in which one or two CH groups per ring may also be replaced by C═O, N, O or S and one or two CH₂ groups in a condensed ring system may also be replaced by C═O or C═CH₂,
Z¹ stands for CR⁷R⁸ or a single bond,
X¹ to X⁴ are each selected, independently of one another, from C—R¹, O, N or S, where 2 adjacent radicals from X¹ to X⁴ together may also stand for an unsubstituted or mono- or polysubstituted ring or condensed ring systems having 6 to 18 C atoms, of which at least one ring preferably has aromatic character, in which one or two CH groups per ring may also be replaced by C═O, N, O or S and one or two CH₂ groups in a condensed ring system may also be replaced by C═O or C═CH₂,
R¹ is selected from H,
   straight-chain or branched C₁- to C₂₀-alkoxy groups, where the alkyl chains may each also be interrupted by oxygen or nitrogen,
   straight-chain or branched C₁- to C₂₀-alkyl groups, where the alkyl chains may each also be interrupted by oxygen or nitrogen,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chains may each also be interrupted by oxygen or nitrogen,
   straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen,
   straight-chain or branched C₁- to C₂₀-alkylamino groups,
   straight-chain or branched C₁- to C₂₀-dialkylamino groups,
   or R¹ stands for a carboxylic acid, phosphoric acid, sulfonic acid, sulfuric acid or sulfone function, which may optionally be esterified or alkylated with straight-chain or branched C₁- to C₂₀-alkyl groups or straight-chain or branched C₃- to C₂₀-alkenyl groups,
R⁷ and R⁸ are each selected, independently of one another, from H, OH, straight-chain or branched C₁- to C₂₀-alkoxy groups, straight-chain or branched C₁- to C₂₀-alkyl groups, straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen,
   straight-chain or branched C₁- to C₂₀-hydroxyalkoxy groups, where the hydroxyl group(s) may be bonded to a primary or secondary carbon atoms of the chain and furthermore the alkyl chain may also be interrupted by oxygen,
      or salts thereof.

The compounds of the formula (3) are preferably selected from the compounds of the formula (3a) and/or (3b)

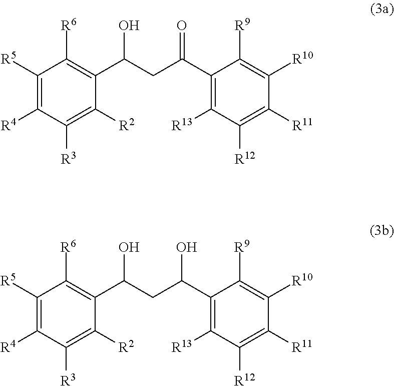

where
R⁴ and R¹¹ each, independently of one another, denote H, straight-chain or branched C1- to C20-alkyl group, straight-chain or branched C1- to C20-alkoxy group or straight-chain or branched C1- to C20-dialkylamino group, R⁶ denotes H or a carboxylic acid, phosphoric acid, sulfonic acid, sulfuric acid or sulfone function, which may be esterified or alkylated with straight-chain or branched C1- to C20-alkyl groups or straight-chain or branched C3- to C20-alkenyl groups and
R², R³, R⁵, R⁹, R¹⁰, R¹² and R¹³ denote H.

The compounds of the formula (3a) and (3b) are particularly preferably selected from the compounds comprising

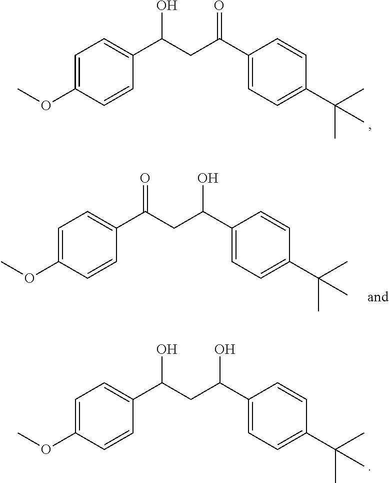

The preparations described, which comprise in accordance with the invention at least one compound of the formula (1) and/or (2), may furthermore also comprise coloured pigments, where the layer structure of the pigments is not limited.

The coloured pigment should preferably be skin-coloured or brownish on use of 0.5 to 5% by weight. The choice of a corresponding pigment is familiar to the person skilled in the art.

Preferred preparations comprise UV filters besides the at least one compound of the formula (1) and/or (2) and the other optional ingredients.

Organic UV filters, so-called hydrophilic or lipophilic sun-protection filters, which are effective in the UVA region and/or UVB region and(/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl) benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: ethylhexyltriazone, for example marketed under the name “Uvinul T150” by BASF, diethylhexylbutamidotriazone, for example marketed under the name “Uvasorb HEB” by Sigma 3V, 2,4,6-tris(diisobutyl 4′-aminobenzalmalonate)-s-triazine or 2,4,6-tris(biphenyl)-1,3,5-triazine. marketed as Tinosorb A2B by BASF, 2,2′-[6-(4-methoxyphenyl)-1,3,5-triazine-2,4-diyl]bis[5-(2-ethylhexyl)oxy]phenol, marketed as Tinosorb S by BASF, N2,N4-bis[4-[5-(1,1-dimethylpropyl)-2-benzoxazolyl]phenyl]-N6-(2-ethylhexyl)-1,3,5-triazine-2,4,6-triamine marketed as Uvasorb K 2A by Sigma 3V.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl) imino]-6-(2-ethylhexyl)imino-1,3,5-triazine, for example marketed by Sigma 3V under the name Uvasorb K2A, and mixtures comprising this.

Piperazine derivatives, such as, for example, the compound

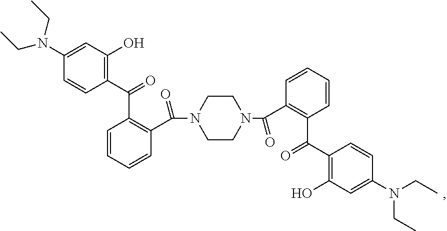 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

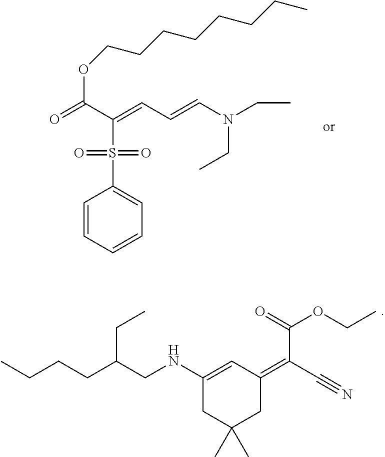

It is also possible to use UV filters based on polysiloxane copolymers having a random distribution in accordance with the following formula, where, for example, a=1.2; b=58 and c=2.8:

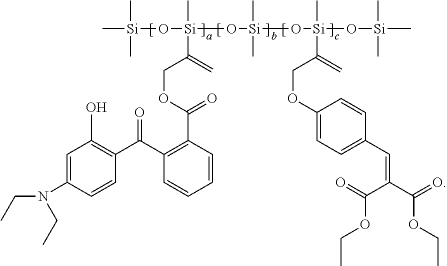

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidenedicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-bis[5-1 (dimethylpropyl)benzoxazol-2-yl(4-phenyl) imino]-6-(2-ethylhexyl)imino-1,3,5-triazine and mixtures thereof.

These organic UV filters are generally incorporated into formulations in an amount of 0.01 percent by weight to 20 percent by weight, preferably 1% by weight—10% by weight.

Besides the compounds of the formula (1) and/or (2) and the optional organic UV filters, as described above, preferred preparations may comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex® T-2000, Eusolex®T-AQUA, Eusolex®T-AVO, Eusolex®T-OLEO), zinc oxides (for example Sachtotec®), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FF-Pharma.

It may furthermore be preferred for the preparations to comprise inorganic UV filters which have been aftertreated by conventional methods, as described, for example, in Cosmetics & Toiletries, February 1990, Vol. 105, pp. 53 64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
Aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
Aftertreated micronised titanium dioxides with aluminium oxide and/or aluminium stearate/laurate aftertreatment, such as, for example, Microtitanium Dioxide MT 100 T from Tayca, Eusolex T-2000 from Merck,
Aftertreated micronised titanium dioxides with iron oxide and/or iron stearate aftertreatment, such as, for example, the product “Microtitanium Dioxide MT 100 F” from Tayca,
Aftertreated micronised titanium dioxides with silicon dioxide, aluminium oxide and silicone aftertreatment, such as, for example, the product “Microtitanium Dioxide MT 100 SAS”, from Tayca,
Aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Microtitanium Dioxide USP Grade Hydrophobic” from Color Techniques.

The combination with the following products may furthermore also be advantageous:
Untreated zinc oxides, such as, for example, the product Z-Cote from BASF (Sunsmart), Nanox from Elementis
aftertreated zinc oxides, such as, for example, the following products:
   “Zinc Oxide CS-5” from Toshibi (ZnO aftertreated with polymethylhydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilesquioxane);
   Untreated cerium oxide micropigment, for example with the name “Colloidal Cerium Oxide” from Rhone Poulenc
   Untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide. zinc oxide mixtures, such as, for example, the product UV-Titan M261 from Sachtleben.

These inorganic UV filters are generally incorporated into the preparations in an amount of 0.1 percent by weight to 25 percent by weight, preferably 2% by weight-10% by weight.

By combination of one or more of the said compounds having a UV filter action, the protective action against harmful effects of the UV radiation can be optimised.

All said UV filters can also be employed in encapsulated form. In particular, it is advantageous to employ organic UV filters in encapsulated form.

The capsules in preparations to be employed in accordance with the invention are preferably present in amounts which ensure that the encapsulated UV filters are present in the preparation in the percent by weight ratios indicated above.

The protective action of preparations against oxidative stress or against the effect of free radicals can be improved if the preparations comprise one or more antioxidants, the person skilled in the art being presented with absolutely no difficulties in selecting antioxidants which act suitably quickly or with a time delay.

Preference is therefore given to preparations which comprise at least one further antioxidant.

There are many proven substances known from the specialist literature which can be used as antioxidants, for example amino acids (for example glycine, histidine, tyrosine, tryptophan) and derivatives thereof, imidazoles, (for ecample urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA, pentasodium ethylenediamine tetramethylene phosphonate and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the formulae A or B

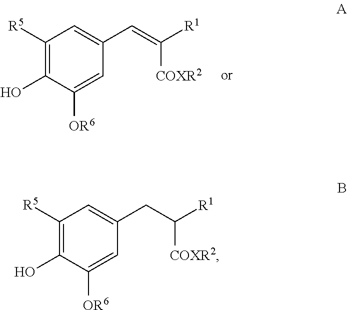

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H, linear or branched alkyl having 1 to 8 C atoms or linear or branched alkoxy having 1 to 8 C atoms and
R⁶ denotes linear or branched alkyl having 1 to 8 C atoms,
   preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with the preparations according to the invention in in amounts of 0.1 to 20% by weight, preferably in amounts of 0.1 to 10% by weight.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology & Medicine 2001, 31(7), 869-881, have investigated the pH dependence of the antioxidant action of hydroxyflavones. Quercetin exhibits the highest activity amongst the structures investigated over the entire pH range.

Preferred preparations may likewise comprise at least one further cosmetic active compound, for example selected from anti-ageing, anti-wrinkle, antidandruff, anti-acne active compounds, antimicrobial active compounds, anti-inflammatory active compounds, anti-cellulite active compounds, self-tanning substances, skin-lightening active compounds, active compounds for improving the moisture content of the skin (skin moisture regulators) or vitamins.

The self-tanning substances can be either a self-tanner which reacts with the amino acids of the skin in the sense of a Maillard reaction or via a Michael addition, or a so-called melanogenesis promoter or propigmentation active compound which promotes the natural tanning of the skin.

Advantageous self-tanning substances which can be employed are, inter alia:

1,3-dihydroxyacetone, glycerolaldehyde, hydroxymethylglyoxal, γ-dialdehyde, erythrulose, 6-aldo-D-fructose, ninhydrin, 5-hydroxy-1,4-naphtoquinone (juglone) or 2-hydroxy-1,4-naphtoquinone (lawsone). Very particular preference is given to 1,3-dihydroxyacetone, erythrulose or a combination thereof.

Propigmentation substances can in principle be all active compounds known to the person skilled in the art. Examples thereof are glycyrrhetinic acid, melanocyte-stimulating hormone (alpha-MSH), peptide analogues, thymidine dinucleotides, L-tyrosine and esters thereof or bicyclic monoterpenediols (described in Brown et al., Photochemistry and Photobiology B: Biology 63 (2001) 148-161).

The at least one self-tanning substance is preferably present in the preparation in an amount of 0.01 to 20% by weight, particularly preferably in an amount of 0.5 to 15% by weight and very particularly preferably in an amount of 1 to 8% by weight, based on the total amount of the preparation.

Preparations having self-tanner properties, in particular those which comprise dihydroxyacetone, tend towards malodours on application to the human skin, which are thought to be caused by degradation products of dihydroxyacetone itself or by products of side reactions and which are perceived as unpleasant by some users. It has been found that these malodours are prevented on use of formaldehyde scavengers and/or flavonoids. The preparation according to the invention may therefore preferably also comprise formaldehyde scavengers and optionally flavonoids for improving the odour.

Suitable anti-ageing active compounds, in particular for skin-care preparations, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N.-acetylornithine, trimethylamine N-oxide di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof.

Additionally, anti-aging active compounds which can be used are products from Merck, such as, for example, 5,7-dihydroxy-2-methylchromone, marketed under the trade name RonaCare® Luremine, RonaCare® Isoquercetin, RonaCare® Tilirosid or RonaCare® Cyclopeptide 5.

The preparations may also comprise one or more skin-lightening active compounds or synonymously depigmentation active compounds. Skin-lightening active compounds can in principle be all active compounds known to the person skilled in the art. Suitable for combination are commercially available melanogenesis inhibitors, such as, for example, ascorbic acid and derivatives thereof, aloesin, niacinamide, emblica, elagic acid, liquorice extract, mulberry extract, kojic acid, liquorice extract, rucinol, hydroquinone and derivatives thereof, azelaic acid, arbutin, magnesium ascorbyl phosphate, pantothenic acid and derivatives or salts thereof, glucosamine and derivatives thereof, mercaptoamines, hinokitol, tocopherols, ubiquinones, glabridin, 4-hydroxyanisole, 4-tert-butylphenol, resveratrol, 4-S-cystaminylphenol, glutathione, lactic acid or the like. Preferred examples of compounds having skin-lightening activity are hydroquinone, niacinamide, ascorbic acid and physiologically acceptable salts thereof, kojic acid, arbutin, aloesin, azelaic acid, elagic acid or rucinol. Preferred examples of extracts having skin-lightening activity are liquorice extract, mulberry extract or emblica.

The preparations to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the flavonoid-containing premixes or preparations in ranges from 0.01 to 5.0% by weight, based on the total weight.

The retinoids described are at the same time also effective anti-cellulite active compounds. A likewise known anti-cellulite active compound is caffeine.

Substances which serve for the maintenance and/or improvement of the moisture content of the skin can, without this being regarded as a restriction, also be, inter alia, substances which belong to the so-called natural moisturising factors, such as, for example, 2-oxopyrrolidine 5-carboxylic acid.

The preparation constituents mentioned can be incorporated in the usual manner, with the aid of techniques which are well known to the person skilled in the art.

Preparations are suitable for external application, for example as cream or milk (O/W, W/O, O/W/O, W/O/W), as lotion or emulsion, in the form of oily/alcoholic, oily/aqueous or aqueous/alcoholic gels or solutions can be sprayed onto the skin. They can be in the form of solid sticks or formulated as aerosol.

The following, for example, may be mentioned as application form of the preparations to be employed: solutions, suspensions, emulsions, PIT emulsions, pastes, ointments, gels, creams, lotions, powders, soaps, surfactant-containing cleansing preparations, oils, aerosols and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexane-carbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of substances:

mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty bodies, as well as water and an emulsifier, as is usually used for such a type of formulation.

In a preferred embodiment, the preparations to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The cosmetic and dermatological preparations can be in various forms. Thus, they can be, for example, a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or also an aerosol. It is also advantageous to administer ectoins in encapsulated form, for example in collagen matrices and other conventional encapsulation materials, for example as cellulose encapsulations, encapsulated in gelatine, wax matrices or liposomally. In particular, wax matrices, as described in DE A-43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laurethyl-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention: fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty bodies, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, use is generally made of the customary propellants, such as alkanes, fluoroalkanes and chlorofluoroalkanes, preferably alkanes.

The present invention furthermore also relates to compounds of the formula (1) or (2)

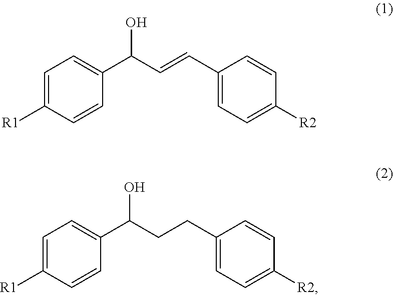

where R1 and R2 stand, independently of one another, for hydrogen, C1- to C12-alkoxy or C1- to C12-alkyl,
where compounds of the formula (1) in which

### R1=R2=H (1l),

R1=R2=methyl,
R1=R2=ethyl,
R1=R2=i-butyl,
R1=R2=methoxy (1c),
R1=methyl, R2=H,
R1=H, R2=methyl,
R1=ethyl, R2=H,
R1=n-heptyl, R2=H,
R1=methoxy, R2=H (1e),
R1=H, R2=methoxy (1f),
R1=methyl, R2=methoxy or
R1=methoxy, R2=methyl,
are excluded
and where compounds of the formula (2) in which

### R1=R2=H (2l),

R1=R2=methyl,
R1=R2=methoxy (2c),
R1=methyl, R2=H,
R1=H, R2=methyl,
R1=ethyl, R2=H,
R1=i-propyl, R2=H (2i),
R1=t-butyl, R2=H (2g),
R1=H, R2=t-butyl (2h),
R1=methoxy, R2=H (2e),
R1=H, R2=methoxy (2f),
R1=H, R2=butoxy,
R1=ethyl, R2=methyl or
R1=methyl, R2=methoxy,
are excluded.

Preferred embodiments of the radicals R1 and R2 are defined as described above. Particular preference is given to compounds of the formula (1) and (2) selected from the compounds of the formula (1a), (1b), (1d), (1g), (1 h), (1i), (1j), (1k), (1m), (1n), (1o), (1p), (2a), (2b), (2d), (2j), (2k), (2m), (2n), (2o) and (2p), where the said compounds are as defined above. Very particular preference is given to the compounds (1a), (1b), (2a) and (2b), in particular (1a) and (2a).

The present invention furthermore also relates to a process for the preparation of a compound of the formula (1) or (2), as defined above, characterised in that a compound of the formula (1-A) or (2-A)

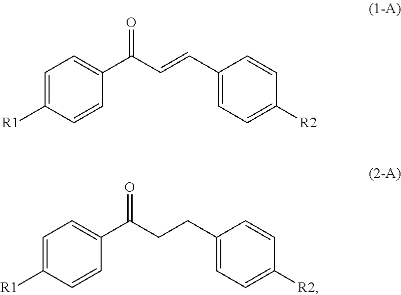

where the radicals R1 and R2 are as defined for formula (1) or formula (2), is converted into a compound of the formula (1) or (2) by catalytic hydrogenation.

A compound of the formula (2-A) is converted here into a compound of the formula (2). A compound of the formula (1-A) can be converted by catalytic hydrogenation both into a compound of the formula (1) and also into a compound of the formula (2).

With respect to details concerning the preparation process, reference is made to the comments above.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications indicated above and below is incorporated into this application by way of reference. The percent by weight ratios of the individual ingredients in the preparations of the examples expressly belong to the disclosure content of the description and can therefore be utilised as features.

The examples of the subject-matter according to the invention which are indicated below serve merely for illustration and in no way narrow the present invention at all. In addition, the invention described can be carried out throughout the range claimed. All compounds or components which can be used in the preparations are either known and commercially available or can be synthesised by known methods. In general, the INCI names of the raw materials used are indicated (the INCI names are by definition indicated in English).

### EXAMPLES

### Example 1

### Synthesis of 3-(4-tert-butyl phenyl)-1-(4-methoxyphenyl)prop-2-en-1-ol (substance 1a)

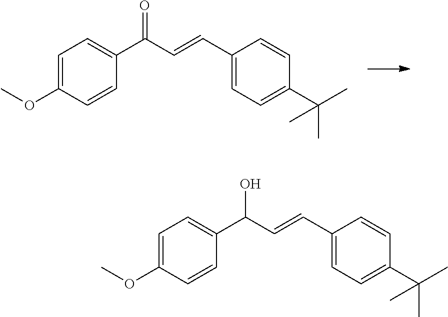

1 mmol of E-3-(4-tert-butylphenyl)-1-(4-methoxyphenyl)propenone is dissolved in a mixture of 15.0 ml of 2-propanol and 5.0 ml of N,N-dimethylformamide. 10.0 mg of ruthenium(II) catalyst and 6.0 mg of potassium tertbutoxide are subsequently added. The hydrogenation is carried out using hydrogen 3.0 at 0° C. and 8 bar pressure. When the hydrogenation is complete, the catalyst is separated off by filtration. The filtrate is freed from solvent in vacuo, and the residue is washed. The organic phase is dried over sodium sulfate, and the solvent is removed in vacuo. The purification is carried out by filtration through silica gel, giving analytically pure product.

### Example 2

### Synthesis of 3-(4-tert-butylphenyl)-1-(4-methoxyphenyl)propan-1-ol (substance 2a)

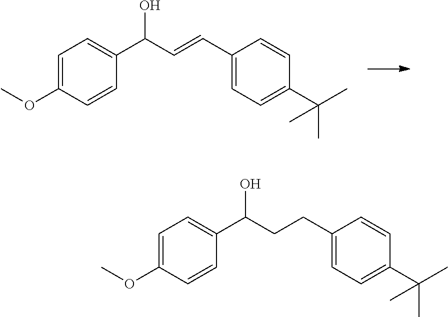

20 ml of tetrahydrofuran (THF) are added to 1 mmol of E-3-(4-tert-butyl phenyl)-1-(4-methoxyphenyl)prop-2-en-1-ol and dissolved. After addition of the catalyst 5% Pd/C (5% Pd/C (56% of water; Merck: Art. No. 275175; 0.54 g/mmol)), the hydrogenation is carried out using hydrogen 3.0 at room temperature and atmospheric pressure. The catalyst is separated off by filtration. The filtrate is freed from solvent in vacuo, and the residue is washed. The organic phase is dried over sodium sulfate, and the solvent is removed in vacuo. The purification is carried out by filtration through silica gel, giving analytically pure product.

### Example 3

Determination of the reducing (=antioxidative) property of E-1,3-diphenyl-2-propen-1-ol (substance 1l): 14 mg of E-1,3-diphenyl-2-propen-1-ol (substance 1l) are dissolved in 10 g of MTB ether. Both the substance solution and also the pure solvent are applied to a TLC plate which has been sprayed in advance with potassium permanganate solution (TLC silica gel 60F254 plate; 5 g of potassium permanganate and 20g of NaOH in 500 ml of dist. water). The potassium permanganate solution decolourises in the region of the substance application 30 seconds after the substance application, whereas the solvent applied for comparison does not decolourise the violet permanganate coloration.

### Example 4

Determination of the reducing (=antioxidative) property of 3-(4-tert-butyl phenyl)-1-(4-methoxyphenyl)prop-2-en-1-ol (substance 1a) and of 3-(4-tert-butylphenyl)-1-(4-methoxyphenyl)propan-1-ol (substance 2a): a little substance is dissolved in MTB ether and applied to a TLC plate (TLC silica gel 60F254 plate). The plate is chromatographed with heptane/ethyl acetate 2/1. After subsequent spraying with freshly prepared potassium permanganate solution, the spray solution decolorises at the substance spots (4 g of NaOH in 96 g of H₂O, addition of 1g of KMnO₄). The Rf values are at 0.61 (substance 2a) and 0.35 (substance 1a).

### Example 5

### Preparations

Illustrative recipes for cosmetic preparations are indicated below. Corresponding preparations can be prepared in the same way with all compounds according to the invention.

In particular, the substances (1a), (1b) and (1l) used in following recipe examples can be replaced in full or part by the corresponding substances (2a), (2b) and (2l). Furthermore, substances according to the invention used may be present in isomeric form. This includes enantiomers and also double-bond isomers. For example, the E double-bond isomers may be replaced in full or part by Z double-bond isomers.

The INCI names of the commercially available compounds are indicated. UV-Pearl, OMC stands for the preparation having the INCI name: Water (for EU: Aqua), Ethylhexyl Methoxycinnamate, Silica, PVP, Chlorphenesin, BHT. This preparation is commercially available from Merck KGaA, Darmstadt, under the name Eusolex®UV Pearl™ OMC.

The other UV-Pearl indicated in the tables each have an analogous composition, with OMC replaced by the UV filters indicated.

TABLE 1
W/O emulsions (numbers in % by weight)

| | 1-1 | 1-2 | 1-3 | 1-4 | 1-5 | 1-6 | 1-7 | 1-8 | 1-9 | 1-10 |
|---|---|---|---|---|---|---|---|---|---|---|
| Titanium dioxide | | 2 | 5 | | | | | | | 3 |
| E-3-(4-tert-Butyl phenyl)- | 5 | 3 | 2 | 1 | 0.5 | | | | | |
| 1-(4-methoxyphenyl)- | | | | | | | | | | |
| prop-2-en-1-ol (1a) | | | | | | | | | | |
| E-1,3-Diphenyl-2-propen- | | | | | 0.5 | 1 | 2 | 3 | 4 | 5 |
| 1-ol (1l) | | | | | | | | | | |
| Zinc oxide | | | | | | | | 5 | 2 | |
| UV-Pearl, OMC | 30 | 15 | 15 | 15 | 15 | 15 | 15 | 15 | 15 | 15 |
| Polyglyceryl-3-Dimerate | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 |
| Caprylic/Capric | 7 | 7 | 7 | 7 | 7 | 7 | 7 | 7 | 7 | 7 |
| Triglyceride | | | | | | | | | | |
| Hexyl Laurate | 4 | 4 | 4 | 4 | 4 | 4 | 4 | 4 | 4 | 4 |
| PVP/Eicosene | 2 | 2 | 2 | 2 | 2 | 2 | 2 | 2 | 2 | 2 |
| Copolymer | | | | | | | | | | |
| Propylene Glycol | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 |
| Propylparabene | 0.05 | 0.05 | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 | 0.15 | 0.15 |
| Water | to 100 | to 100 | to 100 | to 100 | to 100 | to 100 | to 100 | to 100 | to 100 | to 100 |

| | 1-11 | 1-12 | 1-13 | 1-14 | 1-15 | 1-16 | 1-17 | 1-18 |
|---|---|---|---|---|---|---|---|---|
| Titanium dioxide | 3 | | 2 | | 3 | | 2 | 5 |
| Benzylidene malonate polysiloxane | | 1 | 0.5 | | | | | |
| E-3-(4-tert-Butyl phenyl)-1-(4- | 2 | | | 1 | 1 | 2 | | |
| methoxyphenyl)prop-2-en-1-ol (1a) | | | | | | | | |
| E-1,3-Diphenyl-2-propen-1-ol (1l) | | | 2 | 1 | 1 | | | 2 |
| Polyglyceryl-3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Tocopherol | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparabene | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 |
| Dicocoyl Pentyerythrityl Citrate (and) | | | | | 6 | 6 | 6 | 6 |
| Sorbitan Sesquioleate (and) Cera Alba | | | | | | | | |
| (and) Aluminium Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerin | | | | | 5 | 5 | 5 | 5 |
| Allantoin | | | | | 0.2 | 0.2 | 0.2 | 0.2 |
| water | to 100 | to 100 | to 100 | to 100 | to 100 | to 100 | to 100 | to 100 |

| | 1-19 | 1-20 | 1-21 | 1-22 | 1-23 | 1-24 | 1-25 | 1-26 | 1-27 | 1-28 | 1-29 |
|---|---|---|---|---|---|---|---|---|---|---|---|
| Titanium dioxide | | 2 | 5 | | | | | | | 3 | 3 |
| Benzylidene malonate | | | | 1 | | | | | 1 | 1 | |
| polysiloxane | | | | | | | | | | | |
| Methylene Bis-Benztriazolyl | | | | | | 1 | 2 | 1 | | | 1 |
| Tetramethylbutylphenol | | | | | | | | | | | |
| Zinc oxide | | | | | | | | 5 | 2 | | |
| E-3-(4-tert-Butyl phenyl)-1-(4- | 0.5 | 1.0 | 1.5 | 0.5 | 0.5 | 0.5 | | 2.0 | 0.5 | | 2.0 |
| methoxyphenyl)prop-2-en-1-ol | | | | | | | | | | | |
| (1a) | | | | | | | | | | | |
| E-1-(4-tert-Butyl phenyl)-3-(4- | | | 1.5 | | 1.0 | | 1.0 | | | 2.0 | |
| methoxyphenyl)prop-2-en-1-ol | | | | | | | | | | | |
| (1b) | | | | | | | | | | | |
| UV-Pearl, OCR | | 10 | | | | | | | | | 5 |
| UV-Pearl, | | | 10 | | | | | | | | |
| EthylhexylDimethylPABA | | | | | | | | | | | |
| UV-Pearl, Homosalate, BP-3 | | | | | | | | | 10 | | |
| UV-Pearl, Ethylhexyl salicylate, | | | | | | | | | | 10 | |
| BP-3 | | | | | | | | | | | |
| BMDBM | | | | | | | | | | | 2 |
| UV-Pearl OMC, | 25 | | | | | | | | | | |
| 4-Methylbenzylidene Camphor | | | | | | | | | | | |
| Polyglyceryl-3-Dimerate | 3 | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 | 0.5 |
| Phenethyl 3,4-dihydroxyphenyl- | 0.5 | 0.5 | 0.5 | 0.5 | 0.5 | 0.5 | 0.5 | 0.5 | 0.5 | 0.5 | 0.5 |
| propionate | | | | | | | | | | | |
| Propylparabene | 0.05 | 0.05 | 0.05 | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 | 0.15 | 0.15 | 0.15 |

| | |
|---|---|
| Water | to 100 |

TABLE 2
O/W emulsions, numbers in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium dioxide | | 2 | 5 | | | | | | | 3 |
| Methylene Bis-Benztriazolyl | | | | | | 1 | 2 | 1 | | |
| Tetramethylbutylphenol | | | | | | | | | | |
| E-3-(4-tert-Butyl phenyl)-1- | 0.5 | 1 | 2 | 3 | 5 | | | | 1 | 1 |
| (4-methoxyphenyl)prop-2- | | | | | | | | | | |
| en-1-ol (1a) | | | | | | | | | | |
| E-1,3-Diphenyl-2-propen-1- | | | | | | 4 | 3 | 2 | 1 | 0.5 |
| ol (1l) | | | | | | | | | | |
| 4-Methylbenzyliden | 2 | | 3 | | 4 | | 3 | | 2 | |
| Camphor | | | | | | | | | | |
| BMDBM | 1 | 3 | | 3 | 3 | | 3 | 3 | 3 | |
| Stearyl Alcohol (and) | 3 | 3 | 3 | 3 | 3 | 3 | 3 | 3 | 3 | 3 |
| Steareth-7 (and) Steareth-10 | | | | | | | | | | |
| Glyceryl Stearate (and) | 3 | 3 | 3 | 3 | 3 | 3 | 3 | 3 | 3 | 3 |
| Ceteth-20 | | | | | | | | | | |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric Triglyceride | 6 | 6 | 6 | 6 | 6 | 6 | 6 | 6 | 6 | 6 |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparabene | 0.05 | 0.05 | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to 100 | to 100 | to 100 | to 100 | to 100 | to 100 | to 100 | to 100 | to 100 | to 100 |

| | 2-11 | 2-12 | 2-13 | 2-14 | 2-15 | 2-16 | 2-17 | 2-18 |
|---|---|---|---|---|---|---|---|---|
| Titanium dioxide | 3 | | 2 | | | | 2 | 5 |
| Benzylidene malonate polysiloxane | | 1 | 0.5 | | | | | |
| E-1-(4-tert-Butyl phenyl)-3-(4- | 0.5 | 1.5 | 0.5 | 1 | | | | |
| methoxyphenyl)prop-2-en-1-ol (1b) | | | | | | | | |
| E-1,3-Diphenyl-2-propen-1-ol (1l) | | | | | 1 | 0.5 | 1.5 | 0.5 |
| 5,6,7-Trihydroxyflavon | 5 | 5 | 5 | 5 | 5 | 5 | 5 | 5 |
| 2-Ethylhexyl 4-hydroxyphenyl- | 1 | 5 | 4 | | 6 | | 7 | |
| propionate | | | | | | | | |
| Zinc oxide | | | 2 | | | | | |
| UV-Pearl, OMC | 15 | 15 | 15 | 30 | 30 | 30 | 15 | 15 |
| 4-Methylbenzyliden Camphor | | | | 3 | | | | |
| BMDBM | | | | 1 | | | | |
| Phenylbenzimidazole Sulfonic Acid | | | | | 4 | | | |
| Stearyl Alcohol (and) Steareth-7 (and) | 3 | 3 | 3 | 3 | | | | |
| Steareth-10 | | | | | | | | |
| Glyceryl Stearate (and) Ceteth-20 | 3 | 3 | 3 | 3 | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric Triglyceride | 6 | 6 | 6 | 6 | 14 | 14 | 14 | 14 |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparabene | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 |
| Tromethamine | | | | | 1.8 | | | |
| Glycerin | | | | | 3 | 3 | 3 | 3 |
| Water | to 100 | to 100 | to 100 | to 100 | to 100 | to 100 | to 100 | to 100 |

| | 2-19 | 2-20 | 2-21 | 2-22 | 2-23 | 2-24 | 2-25 | 2-26 | 2-27 | 2-28 |
|---|---|---|---|---|---|---|---|---|---|---|
| Titanium dioxide | | | | | | | 3 | 3 | | 2 |
| Benzylidene malonate | 1 | 2 | | | | 1 | 1 | | 1 | 0.5 |
| polysiloxane | | | | | | | | | | |
| 7,8,3′,4′- | | | | 1 | 2 | | | | 1 | 1 |
| Tetrahydroxyflavon | | | | | | | | | | |
| E-3-(4-tert-Butyl phenyl)- | 0.5 | 1 | 2 | 3 | 5 | | | 1 | 1 | 1 |
| 1-(4-methoxyphenyl)prop-2- | | | | | | | | | | |
| en-1-ol (1a) | | | | | | | | | | |
| E-1,3-Diphenyl-2-propen- | | | 1 | | | 4 | 3 | 2 | 1 | 0.5 |
| 1-ol (1l) | | | | | | | | | | |
| Zinc oxide | | | | | 5 | 2 | | | | 2 |
| UV-Pearl, OMC | 15 | 15 | 15 | 15 | 15 | 15 | 15 | 15 | 15 | 15 |
| Caprylic/Capric | 14 | 14 | 14 | 14 | 14 | 14 | 14 | 14 | 14 | 14 |
| Triglyceride | | | | | | | | | | |
| Oleyl Oleate | | | | | | | | | | |
| Propylene Glycol | | | | | | | | | | |
| Glyceryl Stearate SE | 6 | 6 | 6 | 6 | 6 | 6 | 6 | 6 | 6 | 6 |
| Stearic Acid | 2 | 2 | 2 | 2 | 2 | 2 | 2 | 2 | 2 | 2 |
| Persea Gratissima | 8 | 8 | 8 | 8 | 8 | 8 | 8 | 8 | 8 | 8 |
| Propylparabene | 0.05 | 0.05 | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 | 0.15 | 0.15 |
| Glyceryl Stearate, | | | | | | | | | | |
| Ceteareth-20, Ceteareth-10, | | | | | | | | | | |
| Cetearyl Alcohol, | | | | | | | | | | |
| Cetyl Palmitate | | | | | | | | | | |
| Ceteareth-30 | | | | | | | | | | |
| Dicaprylyl Ether | | | | | | | | | | |
| Glycerin | 3 | 3 | 3 | 3 | 3 | 3 | 3 | 3 | 3 | 3 |
| Water | to 100 | to 100 | to 100 | to 100 | to 100 | to 100 | to 100 | to 100 | to 100 | to 100 |

TABLE 3
Gels, numbers in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| A = aqueous gel | | | | | | | | | | |
| Titanium dioxide | | 2 | 5 | | | | | | | 3 |
| 5,6,7-Trihydroxyflavone | | | | 1 | 2 | | | | 1 | 1 |
| E-3-(4-tert-Butyl phenyl)- | 0.5 | | 2 | 3 | 5 | | | | 1 | 1 |
| 1-(4-methoxyphenyl)- | | | | | | | | | | |
| prop-2-en-1-ol (1a) | | | | | | | | | | |
| E-1,3-Diphenyl-2-propen- | | | | | | 4 | 3 | 2 | | 0.5 |
| 1-ol (1l) | | | | | | | | | | |
| E-1-(4-tert-Butyl phenyl)- | | | | | | | | 0.5 | 1 | 0.5 |
| 3-(4-methoxyphenyl)- | | | | | | | | | | |
| prop-2-en-1-ol (1b) | | | | | | | | | | |
| Benzylidene malonate | | | 1 | 1 | 2 | | | | 1 | 1 |
| polysiloxane | | | | | | | | | | |
| Methylene Bis- | | 1 | | | | 1 | 2 | 1 | | |
| Benztriazolyl | | | | | | | | | | |
| Tetramethylbutylphenol | | | | | | | | | | |
| Zinc oxide | | | | 2 | | | | 5 | 2 | |
| UV-Pearl, Ethylhexyl | 30 | 15 | 15 | 15 | 15 | 15 | 15 | 15 | 15 | 15 |
| Mehtoxycinnamat | | | | | | | | | | |
| 4-Methylbenzyliden | | | | | 2 | | | | | |
| Camphor | | | | | | | | | | |
| Butylmethoxydibenzoylmethane | | 1 | | | | | | | | |
| Phenylbenzimidazole | | | 4 | | | | | | | |
| Sulfonic Acid | | | | | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric | 3 | 3 | 3 | 3 | 3 | 3 | 3 | 3 | 3 | 3 |
| Triglyceride | | | | | | | | | | |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol | 0.05 | 0.05 | 0.05 | 0.05 | 0.05 | 0.05 | 0.05 | 0.05 | 0.05 | 0.05 |
| (and) Ascorbyl Palmitate | | | | | | | | | | |
| (and) Ascorbic Acid (and) | | | | | | | | | | |
| Citric Acid | | | | | | | | | | |
| Sorbitol | 4 | 4 | 4 | 4 | 4 | 4 | 4 | 4 | 4 | 4 |
| Polyacrylamide (and) | 3 | 3 | 3 | 3 | 3 | 3 | 3 | 3 | 3 | 3 |
| C13-14 Isoparaffin (and) | | | | | | | | | | |
| Laureth-7 | | | | | | | | | | |
| Propylparabene | 0.05 | 0.05 | 0.05 | 0.05 | 0.05 | 0.05 | 0.05 | 0.05 | 0.05 | 0.05 |
| Methylparabene | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to 100 | to 100 | to 100 | to 100 | to 100 | to 100 | to 100 | to 100 | to 100 | to 100 |
| | | | 3-11 | 3-12 | 3-13 | 3-14 | 3-15 | 3-16 | 3-17 | 3-18 |
| a = aqueaous gel | | | | | | a | a | a | a | a |
| Titanium dioxide | | | 3 | | 2 | | | | | |
| Benzylidene malonate polysiloxane | | | | 1 | 0.5 | 1 | 2 | | | |
| Methylene Bis-Benztriazolyl | | | 1 | 1 | 0.5 | | | 1 | 2 | 1 |
| Tetramethylbutylphenol | | | | | | | | | | |
| E-1,3-Diphenyl-2-propen-1-ol (1l) | | | 1 | | 1 | | 1 | | 1 | 0.5 |
| E-3-(4-tert-Butyl phenyl)-1-(4-methoxyphenyl)prop- | | | 0.5 | 1 | | 1 | | 1 | | 1 |
| 2-en-1-ol (1a) | | | | | | | | | | |
| 6,3′,4′-Trihydroxyflavon | | | 1 | 5 | 4 | | 6 | | 7 | |
| Zinc oxide | | | | | 2 | | | | | |
| UV-Pearl, Ethylhexyl | | | 15 | 15 | 15 | 15 | 15 | 15 | 15 | 15 |
| Mehtoxycinnamat | | | | | | | | | | |
| Prunus Dulcis | | | 5 | 5 | 5 | | | | | |
| Tocopheryl Acetate | | | 0.5 | 0.5 | 0.5 | | | | | |
| Caprylic/Capric Triglyceride | | | 3 | 3 | 3 | | | | | |
| Octyldodecanol | | | 2 | 2 | 2 | | | | | |
| Decyl Oleate | | | 2 | 2 | 2 | | | | | |
| PEG-8 (and) Tocopherol (and) Ascorbyl | | | 0.05 | 0.05 | 0.05 | | | | | |
| Palmitate (and) Ascorbic Acid (and) Citric Acid | | | | | | | | | | |
| Sorbitol | | | 4 | 4 | 4 | 5 | 5 | 5 | 5 | 5 |
| Polyacrylamide (and) C13-14 | | | 3 | 3 | 3 | | | | | |
| Isoparaffin (and) Laureth-7 | | | | | | | | | | |
| Carbomer | | | | | | 1.5 | 1.5 | 1.5 | 1.5 | 1.5 |
| Propylparabene | | | 0.05 | 0.05 | 0.05 | | | | | |
| Methylparabene | | | 0.15 | 0.15 | 0.15 | 0.15 | 0.15 | 0.15 | 0.15 | 0.15 |
| Allantoin | | | | | | 0.2 | 0.2 | 0.2 | 0.2 | 0.2 |
| Tromethamine | | | | | | 2.4 | 2.4 | 2.4 | 2.4 | 2.4 |
| Water | | | to 100 | to 100 | to 100 | to 100 | to 100 | to 100 | to 100 | to 100 |
| | 3-19 | 3-20 | 3-21 | 3-22 | 3-23 | 3-24 | 3-25 | 3-26 | 3-27 | 3-28 |
| 7,8,3′,4′- | | | | 1 | 2 | | | | 1 | 1 |
| Tetrahydroxyflavone | | | | | | | | | | |
| E-1,3-Diphenyl-2-propen- | 0.5 | 1 | 2 | 3 | 5 | | | | 1 | 1 |
| 1-ol (1l) | | | | | | | | | | |
| E-3-(4-tert-Butyl phenyl)- | | | | | | 4 | 3 | 2 | 1 | 0.5 |
| 1-(4-methoxyphenyl)prop-2-en- | | | | | | | | | | |
| 1-ol (1a) | | | | | | | | | | |
| UV-Pearl, OMC | 30 | 30 | 15 | 15 | 15 | 11 | 12 | 15 | 15 | 15 |
| Phenylbenzimidazole | | 4 | 4 | | | | | | | |
| Sulfonic Acid | | | | | | | | | | |
| Sorbitol | 5 | 5 | 5 | 5 | 5 | 5 | 5 | 5 | 5 | 5 |
| Carbomer | 1.5 | 1.5 | 1.5 | 1.5 | 1.5 | 1.5 | 1.5 | 1.5 | 1.5 | 1.5 |
| Propylparabene | | | | | | | | | | |
| Methylparabene | 0.15 | 0.15 | 0.15 | 0.15 | 0.15 | 0.15 | 0.15 | 0.15 | 0.15 | 0.15 |
| Allantoin | 0.2 | 0.2 | 0.2 | 0.2 | 0.2 | 0.2 | 0.2 | 0.2 | 0.2 | 0.2 |
| Tromethamine | 2.4 | 4.2 | 4.2 | 2.4 | 2.4 | 2.4 | 2.4 | 2.4 | 2.4 | 2.4 |
| Water | to 100 | to 100 | to 100 | to 100 | to 100 | to 100 | to 100 | to 100 | to 100 | to 100 |

| | 3-29 | 3-30 | 3-31 | 3-32 | 3-33 | 3-34 | 3-35 | 3-36 |
|---|---|---|---|---|---|---|---|---|
| E-3-(4-tert-Butyl phenyl)-1-(4- | 0.5 | 1 | 2 | 3 | 5 | | | |
| methoxyphenyl)prop-2-en-1-ol (1a) | | | | | | | | |
| E-1-(4-tert-Butyl phenyl)-3-(4-methoxyphenyl)prop- | | | | | | 4 | 3 | 2 |
| 2-en-1-ol (1b) | | | | | | | | |
| E-1,3-Diphenyl-2-propen-1-ol (1l) | 0.5 | 0.5 | 0.5 | | | | | |
| 5,6,7-Trihydroxyflavon | 1 | 5 | 4 | | 6 | | 7 | |
| UV-Pearl, OMC | 15 | 10 | | 10 | 10 | 10 | 15 | 10 |
| UV-Pearl, OCR | | | 10 | | | | | |
| UV-Pearl, OMC, Methylene Bis-Benztriazolyl | | 7 | | 6 | | | | |
| Tetramethylbutylphenol | | | | | | | | |
| UV-Pearl, Ethylhexyl salicylate, BMDBM | | | 10 | | | | | |
| Disodium Phenyl Dibenzimidazole | | 3 | | | | 3 | | 3 |
| Tetrasulfonate | | | | | | | | |
| Phenylbenzimidazole Sulfonic Acid | | 2 | | | 2 | 3 | | 3 |
| Prunus Dulcis | 5 | 5 | 5 | | | | | |
| Tocopheryl Acetate | 0.5 | 0.5 | 0.5 | | | | | |
| Caprylic/Capric Triglyceride | 3 | 3 | 3 | | | | | |
| Octyldodecanol | 2 | 2 | 2 | | | | | |
| Decyl Oleate | 2 | 2 | 2 | | | | | |
| PEG-8 (and) Tocopherol (and) Ascorbyl | 0.05 | 0.05 | 0.05 | | | | | |
| Palmitate (and) Ascorbic Acid (and) Citric Acid | | | | | | | | |
| Sorbitol | 4 | 4 | 4 | 5 | 5 | 5 | 5 | 5 |
| Polyacrylamide (and) C13-14 | 3 | 3 | 3 | | | | | |
| Isoparaffin (and) Laureth-7 | | | | | | | | |
| Carbomer | | | | 1.5 | 1.5 | 1.5 | 1.5 | 1.5 |
| Propylparabene | 0.05 | 0.05 | 0.05 | | | | | |
| Methylparabene | 0.15 | 0.15 | 0.15 | 0.15 | 0.15 | 0.15 | 0.15 | 0.15 |
| Allantoin | | | | 0.2 | 0.2 | 0.2 | 0.2 | 0.2 |
| Tromethamine | | | | 2.4 | 2.4 | 2.4 | 2.4 | 2.4 |
| Water | to 100 | to 100 | to 100 | to 100 | to 100 | to 100 | to 100 | to 100 |

### Example 6

### Hair Mascara

| Ingredients | [%] |
|---|---|
| A | |
| PEARLESCENT PIGMENT | 20.00 |
| B | |
| CETEARETH-25 | 1.80 |
| CETEARYLALCOHOL | 5.00 |
| DIMETHICONE | 1.00 |
| PHENOXYETHANOL, BUTYLPARABEN, | 0.50 |
| ETHYLPARABEN, PROPYLPARABEN, | |
| METHYLPARABEN | |
| C | |
| AQUA (WATER) | to 100 |
| POLYQUATERNIUM-16 | 3.0 |
| PROPYLENEGLYCOL | 1.80 |
| E-1,3-DIPHENYL-2-PROPEN-1-OL (1l) | 0.25 |
| E-3-(4-TERT-BUTYL PHENYL)-1-(4- | 1.5 |
| METHOXYPHENYL)PROP-2-EN-1-OL (1a) | |
| D | |
| AQUA (WATER) | 9.50 |
| HYDROXYPROPYLCELLULOSE | 0.50 |
| E | |
| AQUA (WATER) | 9.50 |
| MAGNESIUM ALUMINIUM SILICATE | 0.50 |
| IMIDAZOLIDINYL UREA | 0.30 |

### Preparation Process:

Heat phase B to 75° C., phase C to 80° C. Slowly add phase B to phase C with stirring. Cool to 65° C. with stirring and homogenise. Cool to 40° C. and add phases D, E and F to phase B/C with stirring and homogenise again. Now add the pearlescent pigment with stirring. Cool to room temperature and adjust the pH to 6.0-6.5.

### Example 7

### Conditioner Comprising IR3535®

| Ingredients | [%] |
|---|---|
| A | |
| ETHYLBUTYLACETYLAMINOPROPIONATE | 10.00 |
| PVP/VA COPOLYMER | 4.00 |
| PERFUME | 0.30 |
| QUATERNIUM-80 | 1.0 |
| PEG-40 HYDROGENATED CASTOR OIL | 1.00 |
| ALCOHOL | 15.00 |
| E-1,3-DIPHENYL-2-PROPEN-1-OL (1l)) | 0.5 |
| E-3-(4-TERT-BUTYL PHENYL)-1-(4- | 0.5 |
| METHOXYPHENYL)PROP-2-EN-1-OL (1a) | |
| B | |
| CETRIMONIUM CHLORIDE | 0.50 |
| AQUA (WATER) | to 100 |
| C | |
| COCAMIDOPROPYL BETAINE | 4.00 |

### Preparation Process:

Mix phases A and B separately. Add phase B to phase A with stirring. Add phase C.

### Example 8

### Hair Conditioner Comprising Pearlescent Pigment

| Ingredients | [%] |
|---|---|
| A | |
| PEARLESCENT PIGMENT | 3.00 |
| DISODIUM EDTA | 0.05 |
| AQUA (WATER) | to 100 |
| B | |
| CETEARYLALCOHOL, BEHENTRIMONIUM | 5.00 |
| METHOSULFATE | |
| OCTYLDODECANOL | 1.10 |
| CETYLALCOHOL | 1.00 |
| GLYCERIN | 1.00 |
| BEHENTRIMONIUM CHLORIDE | 0.70 |
| METHOXY PEG/PPG-7/3 AMINOPROPYL | 0.70 |
| DIMETHICONE | |
| QUATERNIUM-80 | 1.0 |
| E-3-(4-TERT-BUTYL PHENYL)-1-(4- | 0.1 |
| METHOXYPHENYL)PROP-2-EN-1-OL (1a) | |
| E-1,3-DIPHENYL-2-PROPEN-1-OL (1l) | 1.0 |
| COMPOUND OF FORMULA IB-IAH | 2.0 |
| C | |
| COCODIMONIUM HYDROXYPROPYLSILIKAMINO | 0.70 |
| ACIDS | |
| PHENOXYETHANOL, BENZOIC ACID, | 0.40 |
| DEHYDROACETIC ACID | |
| CITRIC ACID | 0.20 |
| PERFUME | 0.60 |

### Preparation Process:

Disperse the pearlescent pigment and Titriplex III in the water of phase A. Heat the constituents of phases A and B to 75° C. Add phase B to phase A with stirring and homogenise. Cool to 40° C. and add the constituents of phase C. Cool to 30° C. and homogenise again for about 30 sec. Adjust the pH to 3.6-4.0.

Notes: recommended pearlescent pigments are TIMIRON® silver pigments and TIMIRON® interference pigments from Merck.

1. An antioxidant comprising one or more compounds of the formula (1) and/or (2) 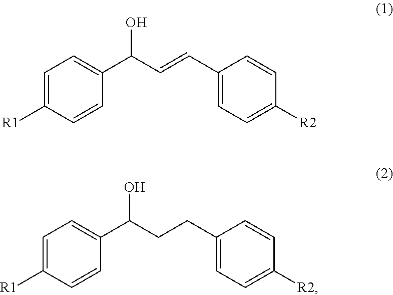
where R1 and R2 stand, independently of one another, for hydrogen, C1- to C12-alkoxy or C1- to C12-alkyl.

2. An antioxidant of claim 1 for prophylaxis against or for combating of oxidative stress.

3. An antioxidant of claim 1 for the care, preservation or improvement of the general condition or appearance of the skin or hair.

4. An antioxidant of claim 1 for prophylaxis against or for combating of time- and/or light-induced ageing processes of the skin or hair.

5. An antioxidant of claim 1 for prophylaxis against or for combating of sebum oxidation and/or acne.

6. An antioxidant according to claim 1, characterised in that R1 stands for hydrogen, C1- to C6-alkyl or C1- to C6-alkoxy.

7. An antioxidant according to claim 6, characterised in that R1 stands for hydrogen, methoxy, isopropyl or t-butyl.

8. An antioxidant according to claim 1, characterised in that R2 stands for hydrogen, C1- to C6-alkyl or C1- to C6-alkoxy.

9. An antioxidant according to claim 8, characterised in that R2 stands for hydrogen, methoxy, isopropyl or t-butyl.

10. An antioxidant according to claim 1, characterised in that formula (1) is selected from compounds of the formulae (1a) to (1p) and formula (2) is selected from compounds of the formulae (2a) to (2p)

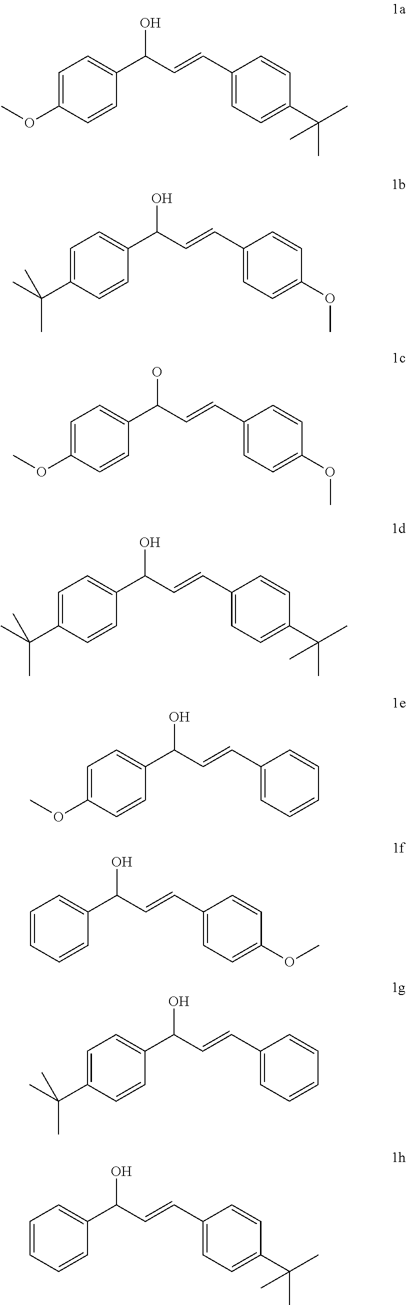

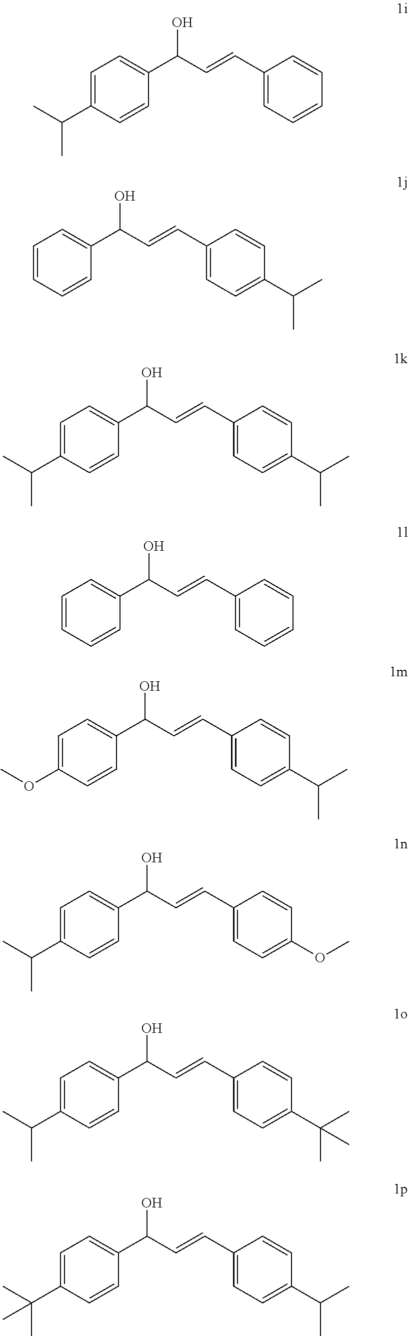

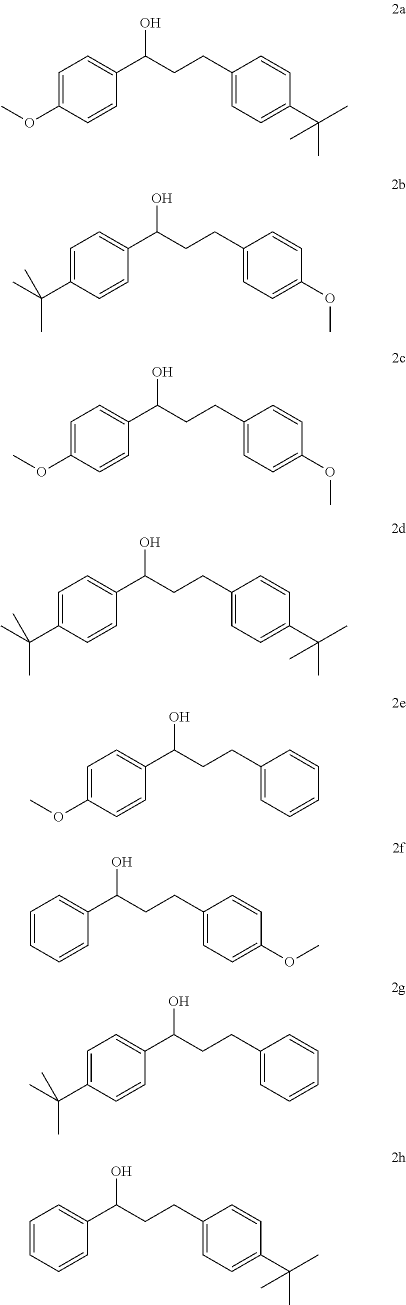

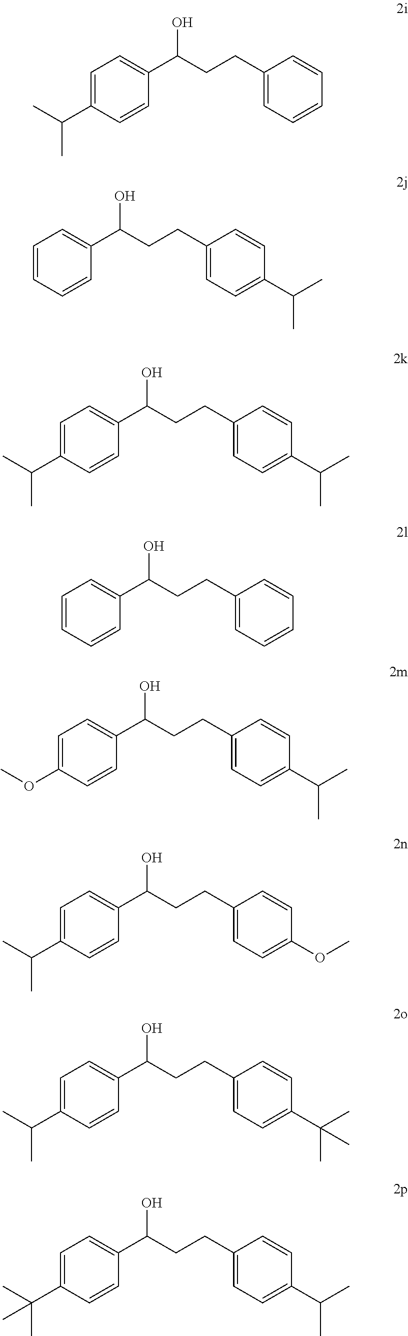

11. Preparation comprising at least one compound of the formula (1) and/or (2) 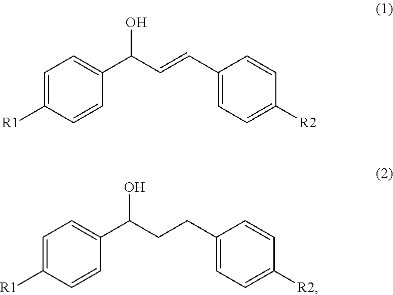
where R1 and R2 stand, independently of one another, for hydrogen, C1- to C12-alkoxy or C1- to C12-alkyl,
and at least one vehicle which is suitable for topical applications.

12. Preparation according to claim 11, characterised in that the at least one compound of the formula (1) or (2) is present in an amount of 0.01 to 20% by weight.

13. Process for the preparation of a preparation according to claim 11, characterised in that the at least one compound of the formula (1) or (2) is mixed with the vehicle which is suitable for topical applications.

14. Compound of the formula (1) or (2) 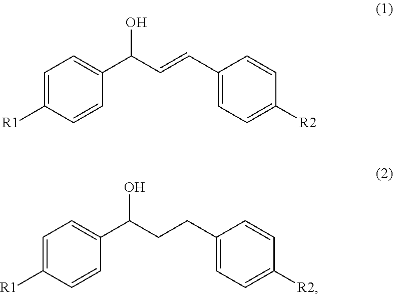
where R1 and R2 stand, independently of one another, for hydrogen, C1- to C12-alkoxy or C1- to C12-alkyl,
where compounds of the formula (1) in which
R1=R2=H (1l),
R1=R2=methyl,
R1=R2=ethyl,
R1=R2=i-butyl,
R1=R2=methoxy (1c),
R1=methyl, R2=H,
R1=H, R2=methyl,
R1=ethyl, R2=H,
R1=n-heptyl, R2=H,
R1=methoxy, R2=H (1e),
R1=H, R2=methoxy (1f),
R1=methyl, R2=methoxy or
R1=methoxy, R2=methyl,
are excluded
and where compounds of the formula (2) in which
R1=R2=H (2l),
R1=R2=methyl,
R1=R2=methoxy (2c),
R1=methyl, R2=H,
R1=H, R2=methyl,
R1=ethyl, R2=H,
R1=i-propyl, R2=H (2i),
R1=t-butyl, R2=H (2g),
R1=H, R2=t-butyl (2h),
R1=methoxy, R2=H (2e),
R1=H, R2=methoxy (2f),
R1=H, R2=butoxy,
R1=ethyl, R2=methyl or
R1=methyl, R2=methoxy,
are excluded.

15. Process for the preparation of a compound of the formula (1) or (2) according to claim 14, characterised in that a compound of the formula (1-A) or (2-A) 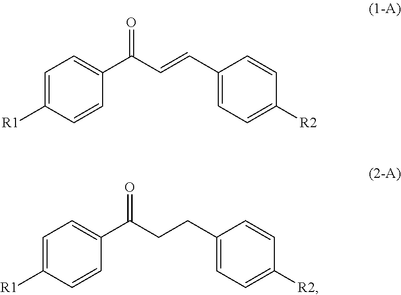
where the radicals R1 and R2 are as defined for formula (1) or formula (2), is converted into a compound of the formula (1) or (2) by catalytic hydrogenation.
